(19) United States
(12) Patent Application Publication
(10) Pub. No.: US 2015/0032088 A1
(43) Pub. Date: Jan. 29, 2015

(54) SUSTAINED DRUG DELIVERY FROM SOLID STATE COMPOSITIONS WITH NANOCHANNEL MEMBRANES

(71) Applicants: The Methodist Hospital Research Institute, Houston, TX (US); Baylor College of Medicine, Houston, TX (US)

(72) Inventors: Alessandro Grattoni, Houston, TX (US); Ganesh Subramanyam Palapattu, Houston, TX (US); Mohit Khera, Houston, TX (US)

(21) Appl. No.: 14/374,094

(22) PCT Filed: Jan. 24, 2013
(86) PCT No.: PCT/US2013/022987

§ 371 Date: Jul. 23, 2014

Related U.S. Application Data
(60) Provisional application No. 61/590,265, filed on Jan. 24, 2012.

(51) Int. Cl. A61M 31/00; A61K 31/198; A61K 31/568

(52) U.S. Cl. CPC A61M 31/002; A61K 31/568; A61K 31/198; A61M 5/14276
USPC 604/522; 604/93.01

(57) ABSTRACT

Implantable devices and methods for delivering pharmaceutical substances into a patient. The implantable device includes the following components: (a) an implant body, (b) a reservoir in the implant body, (c) a heterogeneous composition disposed in the reservoir, which includes a pharmaceutical substance in the solid state contacted by a solution of the pharmaceutical substance, and (d) a nanochannel membrane for delivering the pharmaceutical substance from the reservoir to a patient. Extended, high-quality delivery of low solubility drugs such as hormones and chemotherapeutic agents are achieved.

### FEDERAL FUNDING STATEMENT

This invention was carried out pursuant to Grant No. NNJ06HE06A from the National Aeronautics and Space Administration. The government has certain rights in the invention.

### BACKGROUND

Nanochannel membranes for long term controlled release of drugs from implantable devices have been previously developed. Sharma et al., Expert Opin. Drug Deliv. 3(3):379- 394 (2006); Martin et al., J. Control Release 102(1) 123-133 (2005). Methods have been developed for the fabrication of silicon-based mechanically robust devices with hundreds of thousands of densely packed nanochannels with precisely controlled size and surface properties. Grattoni et al., Lab on a Chip 10:3074-3083 (2010). Sacrificial layer techniques have been used to reproducibly fabricate nanochannels as small as 3 nm. US 2010/0152699.

Moreover, nanoscale fluidics and molecular diffusion in nanochannels have been studied. Cosentino et al., J. Phys. Chem. 109:7358-7364 (2005); Ziemys et al., Journal of Computational Physics 230:5722-5731 (2011). At the nanoscale, molecular interactions with the channel wall dominate the transport of fluids to such an extent that the classical mechanical laws of diffusion (Fick's laws) break down. Thus, nanoscale phenomena can be exploited to achieve the goal of constant release of nanoparticles and therapeutics over periods of time ranging from weeks to months and over a broad range of molecular sizes, at release rates relevant for medical applications. Constant and sustained release has been achieved with a large number of soluble molecules ranging from small molecular weight (MW) peptides such as leuprolide, a LH-RH agonist and common treatment for prostatic cancer, as well as large MW proteins such as bevacizumab, a monoclonal antibody to VEGF widely used in the treatment of metastatic colon cancer and other diseases. See e.g., Grattoni et al., Pharm. Res. 28(2):292-300 (2011). Furthermore, it has been demonstrated both in vitro and in vivo, that Interferon a-2b and lysozyme can be delivered constantly in a healthy rat model for over 6 months. Walczak et al., Nanobiotechnology 1:35-42 (2005).

However, prior art methods for the constant and sustained delivery of pharmaceutical substances as a solution using implantable capsules require the pharmaceutical substances to be soluble. Many pharmaceutical substances have poor solubility, and thus are not suitable to be delivered as a solution for an extended period of time using an implantable capsule. Moreover, some pharmaceutical substances, though soluble, need to be delivered in large quantity to be effective in a patient. It would be difficult to load a sufficient amount of a solution thereof in an implantable capsule for sustained delivery.

A need exists for methods and devices for delivering clinical drugs of solid formulation in patients in a constant and sustained manner for an extended period of time.

### SUMMARY

Embodiments described here include, for example, compositions, articles, devices, methods of making, and methods of using.

One embodiment provides, for example, an implantable device comprising: at least one implant body; at least one reservoir in said implant body; wherein inside the reservoir is disposed at least one pharmaceutical substance in a solid state contacted by at least one solution of said pharmaceutical substance, said solution comprising at least one solvent; and at least one nanochannel membrane for delivering said pharmaceutical substance from the reservoir to a patient.

Another embodiment provides a method for delivering a pharmaceutical substance, comprising: providing at least one implantable device described in the previous paragraph, and implanting said implant into a patient, wherein said pharmaceutical substance is released from the device to contact said patient.

Another embodiment provides, for example, an implantable device comprising: at least one implant body comprising at least one exit port; at least one reservoir in said implant body; wherein inside the reservoir is disposed testosterone in powder or pellet form contacted by at least one solution of the testosterone, said solution comprising at least one solvent; and at least one nanochamel membrane having at least one lateral dimension of 1-200 nm in fluid communication with the reservoir and the exit port for delivering said pharmaceutical substance from the reservoir to the exit port.

Another embodiment provides a method for delivering testosterone, comprising: providing at least one implantable device described in the previous paragraph, and implanting said device into a patient, wherein the testosterone is released from the device to the patient at a rate of 1-10 mg/day.

Another embodiment provides, for example, an implantable device comprising: at least one implant body comprising at least one exit port; at least one reservoir in said implant body; wherein inside the reservoir is disposed thyroxine in powder or pellet form contacted by at least one solution of the thyroxine, said solution comprising at least one solvent; and at least one nanochannel membrane having at least one lateral dimension of 1-200 nm in fluid communication with the reservoir and the exit port for delivering said pharmaceutical substance from the reservoir to the exit port.

Another embodiment provides a method for delivering thyroxine, comprising: providing at least one implantable device described in the previous paragraph, and implanting said device into a patient, wherein the thyroxine is released from the device to the patient at a rate of 50-400 μg/day.

Another embodiment comprises a device comprising: at least one body comprising at least one exit port; at least one reservoir in said implant body; wherein inside the reservoir is disposed at least one pharmaceutical substance in a solid state contacted by at least one solution of said pharmaceutical substance, said solution comprising at least one solvent; and at least one membrane comprising at least one nanochannel, at least one inlet, and at least one outlet, wherein the membrane is in fluid communication with the reservoir and the exit port, to provide delivery of the pharmaceutical substance from the reservoir to the exit port.

Another embodiment provides a method for delivering solid state substance, comprising: determine the daily dose of a pharmaceutical substance to be delivered into a patient; providing a capsule comprising therein said pharmaceutical substance partially in solid state and partially dissolved in a solvent, wherein said capsule comprises a plurality of nanochannels having at least one lateral dimension of 1000 nm or less; implanting said capsule into the patient; releasing said pharmaceutical substance into said patient through said nanochannel, wherein said pharmaceutical substance is released at said daily dose for three months or more; and wherein said capsule cannot be loaded with a sufficient amount of said pharmaceutical substance totally dissolved in said solvent for releasing at said daily dose for three months or more.

Another embodiment is a method comprising: providing at least one implant device comprising at least one implant body comprising at least one reservoir in said implant body;

and optionally at least one nanochannel membrane for delivering a pharmaceutical substance from the reservoir to a patient; and loading said reservoir with at least one pharmaceutical substance in a solid state and at least one solution of said pharmaceutical substance contacting the solid state pharmaceutical composition, said solution comprising at least one solvent.

Methods and devices described here can include one or more of the following advantages for at least one embodiment:

1. The use of a solid degradable drug formulation nullifies the common issue of burst release and allows maintaining a constant and sustained administration of drug. 2. The use of a solid drug formulation allows drugs with low solubility to be delivered in a constant and sustained manner.

3. The use of a solid drug formulation improves the stability of the stored drug, which benefits long term therapeutic applications.

4. The use of a solid drug formulation increases the loading efficacy of the implant and decreases the implant volume needed.

5. The use of a solid drug formulation avoids the potential hazard of drug overdose in the remote case of implant rupture.

6. While not limited by theory, the use of the nanochannel membrane in combination with the implant loaded with solid drug can enable a constant release by means of two possible mechanisms. First, nanoconfinement effect created by the nanochannels is exploited, which can neutralize the initial burst release and the release drop at high percentages of released amount. Second, the nanochannels also work as a dumping system—they maintain the reservoir solution at a steady concentration, which could be the solubility limit of the drug. This will impose a constant concentration gradient across the entire membrane which will act together with the nanoconfinement effect to sustain a constant release of the drug.

7. Methods and devices described here overcomes the limitation of conventional implantable drug delivery systems (e.g. degradable pellets) and can be used for a much longer period of time. For example, methods described here are suitable for constant delivery of testosterone and thyroxine for a period exceeding 1 year (3 times longer duration than implantable pellets).

8. Prior art methods of drug administration are associated with peaks and troughs of drugs levels in the body. Such fluctuations adversely affect drug efficacy and toxicities. Methods and devices described here removes much of such fluctuations and hence may allow the administration of specific drugs (at lower overall amounts) with few side effects. Potential applications include chemotherapy.

9. The use of refillable capsules allows refilling the reservoir without explanting the device.

10. Methods and devices described here reduce compliance issues for treatment extended for long periods of time (e.g. treatment for chronic pathologies). Patients no longer need to volitionally take a drug repeatedly.

11. Methods and devices described here have the potential for artificial gland to replace basal hormone delivery from defective glands of the body (e.g. thyroid).

12. Because of the constant delivery release profile, drug levels are projected to be within the therapeutic window for the vast majority of treatment time. Hence, drug toxicities related to wide variations in drug peak and trough levels can be avoided and overall lesser amounts of total drug will be used.

### BRIEF DESCRIPTION OF THE DRAWINGS

FIG. 1. (A) Perspective view of an exemplary cylindrical-shaped implantable device. (B) Perspective view of an exemplary disc-shaped implantable device. (C) Cross-sectional view of an exemplary implantable device.

FIG. 2. (A) Top view of an exemplary silicon nanochannel membrane. (B) Cross- sectional view of an exemplary custom UV diffusion device for testing drug release through nanochannel membranes.

FIG. 3 shows an exemplary standard curve relating UV absorbance to testosterone concentration.

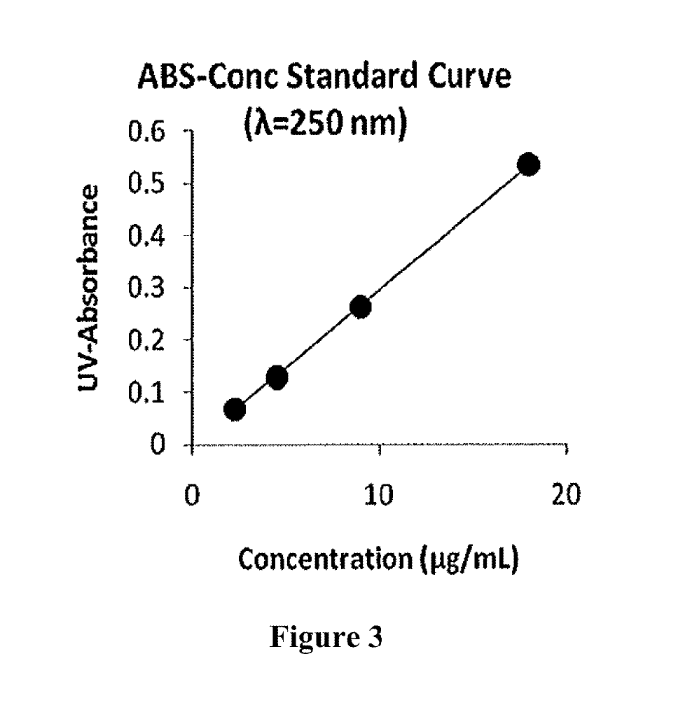

FIG. 4 shows an exemplary delivery curve of testosterone dissolving from solid pellets according to methods and devices described here. Constant delivery of testosterone dissolving from solid pellets through 3 nm nanochannel membrane was recorded for 13 days.

FIG. 5 shows serum testosterone levels in testosterone-deficient men implanted with 10-12 prior art crystalline testosterone pellets, by BMI, beginning at day 1 post-implantation. Red—Normal, BMI<25; Blue—Overweight, BMI 25-30; Green—Obese, BMI>30. All group studied show a decay of testosterone level over time.

FIG. 6 shows exemplary delivery curves of testosterone according to methods described herein. Blue—Constant delivery of testosterone from powder through 3 nm nanochannel membrane was recorded for over 180 days. Red—Constant delivery of testosterone from pellet through 3 nm nanochannel membrane was recorded for over 180 days.

FIG. 7 shows exemplary delivery curves of testosterone according to methods described here. Blue—Constant delivery of testosterone from powder through 40 nm nanochannel membrane was recorded for over 160 days. Red—Constant delivery of testosterone from pellet through 40 nm nanochannel membrane was recorded for over 160 days.

FIG. 8 shows cumulative release curves for levothyroxine from a 3 nm nanochannel membrane. The curves show excellent continuous and constant release of levothyroxine over 17 days from 2 separate experiments (019 and 040). The release pattern corresponds to an approximate daily release rate of 500 micrograms/day. Current conventional human dosing is 50-400 micrograms/day.

### DETAILED DESCRIPTION

### Introduction

Each reference cited herein is incorporated by reference in its entirety.

### Nanochannel Membrane

Nanochannel membranes are known in the art and described in, for example, Sharma et al., Expert Opin. Drug Deliv. 3(3):379-394 (2006); Martin et al., J. Control Release 102(1) 123-133 (2005); Grattoni et al., Lab on a Chip 10:3074-3083 (2010); Grattoni et al., Pharm. Res. 28(2):292-300 (2011); Grattoni et al., Anal. Chem. 83:3096-3103 (2011); and US 2010/0152699, all of which are incorporated herein by reference in their entireties. These refererences also describe how to make the membranes by, for example, microfabrication methods.

The nanochannel membrane can have a plurality of nanochannels. For example, the nanochannel membrane can have 100 nanochannels or more, or 1,000 nanochannels or more, or 10,000 nanochannels or more, or 100,000 nanochannels or more, or 1,000,000 nanochannels or more. The optimal number of nanochannels that allows for the constant release of the required amount of a particular pharmaceutical substance may depends on the size of the nanochannels. In the case of testosterone, the nanochannel membrane may comprise several millions of 3 nm nanochannels, or hundreds of thousands of 200 nm nanochannels.

The nanochannels on the nanochannel membrane can be the same or different. In one embodiment, the nanochannel membrane comprises one set of uniform nanochannels. In another embodiment, the nanochannel membrane comprises at least two sets of nanochannels each having a unique size.

The nanochannel can have at least one lateral dimension of 1,000 nm or less, or 500 nm or less, or 200 nm or less, or 100 nm or less, or 50 nm or less, or 20 nm or less, or 10 nm or less, or 5 nm or less. In a preferred embodiment, the nanochannel has a lateral dimension of 1-200 nm. In a more preferred embodiment, the nanochannel has a lateral dimension of 3- 50 nm. In a particular embodiments, the nanochannel has a lateral dimension of 3 nm; in another particular embodiment, the nanochannel has a lateral dimension of 40 nm.

The nanochannels can be, for example, oriented parallel to the primary plane of the nanochannel membrane. In addition to the nanochannels, the nanochannel membrane can comprise, for example, at least one inlet microchannel and at least one outlet microchannel. The nanochannel can be, for example, in direct communication with both the inlet microchannel and the outlet microchannel. The inlet microchannel can be, for example, in direct communication with the reservoir of a capsule. The outlet microchannel can be, for example, in direct communication with the outside of the capsule, optionally via an exit port.

The nanochannel membrane can be oriented, for example, parallel to the primary plane of the nanochannel membrane. A flow path from the inlet microchannel to the nanochannel to the outlet microchannel can have, for example, a maximum of two changes in direction.

Methods for making the nanochannel membrane described here are exemplified in US 2010/0152699, incorporated by reference in its entirety. The nanochannel membrane can be made of silicon. The nanochannel membrane can also be fabricated with other ceramics including aluminum oxide, titanium oxide, silicon nitride, and silicon carbide. Moreover, metals could be used including gold, platinum, and titanium. Furthermore, polymers such as Teflon, Silicone rubber, PC, and PE, among many others, can be employed.

### Implantable Device/Capsule

Implantable devices and/or capsules are known in the art and described in, for example, Grattoni et al., ASME Mechanical Engineering 133(2):23-26 (2011); Walczak et al., Nanobiotechnology 1:35-42 (2005); Sharma et al., Expert Opin. Drug Deliv. 3(3):379-394 (2006); Martin et al., J. Control Release 102(1) 123-133 (2005); U.S. Pat. No. 5,837,276; U.S. Pat. No. 6,306,420; and US 2010/0152699, all of which are incorporated herein by reference in their entireties. The device can comprise an implant body with walls which can comprise an impermeable material. The implant body can be made of, for example, stainless steel, titanium, polyetheretherketone (PEEK) or other biocompatible materials.

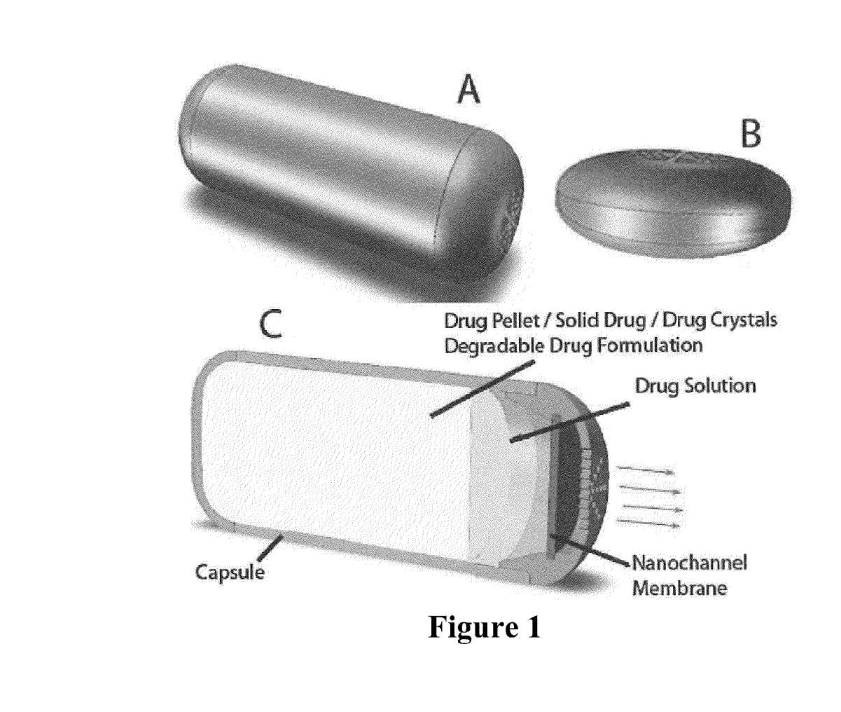

The device or capsule can be of any shape. The capsule can have, for example, a cylindrical body as shown in FIG. 1A. The capsule can have, for example, a disc-like body as shown in FIG. 1B. The diameter to height ratio can be less than one or more than one. The space within the capsule body can be, for example, a reservoir for storing a pharmaceutical composition. The capsule can have an optional first cap for capping the nanochannel membrane. The capsule can have a optional septum of a self-sealing material that permits injection of a pharmaceutical composition inside the capsule body. The capsule can have an optional second cap for capping the septum.

The device or capsule can have, for example, two or more separated reservoirs, each storing a different pharmaceutical composition, and each in communication with a different nanochannel membrane. In this embodiment, two more or pharmaceutical compositions can be delivered using the same implantable capsule.

A cylindrical device or capsule can have, for example, a length and a width. The length of the capsule can be, for example, 1-20 mm, 20-40 mm, 40-60 mm, 60-80 mm, 80-100 mm, 100-120 mm, 120-140 mm, 140-160 mm, 160-180 mm, 180-200 mm, or more than 200 mm. The width of the capsule can be, for example, 0.1-1 mm, 1-2 mm, 2-5 mm, 5-10 mm, 10-15 mm, 15-20 mm, 20-50 mm, 50-100 mm, or more than 100 mm.

A disc-like device or capsule can have, for example, a height and a diameter. The height of the capsule can be, for example, 0.1-1 mm, 1-2 mm, 2-3 mm, 3-4 mm, 4-5 mm, 5-6 mm, 6-7 mm, 7-8 mm, 8-9 mm, 9-10 mm, or more than 10 mm. The diameter of the capsule can be, for example, 0.5-1 mm, 1-5 mm, 5-10 mm, 10-15 mm, 15-20 mm, 20-25 mm, 25-30 mm, 30-50 mm, 50-100 mm, or more than 100 mm

The size of the capsule and the volume of the reservoir can be adapted to fit the amount of a pharmaceutical substance required for the predicted duration of treatment.

The optimal shape of the capsule can be ergonomic with respect of the implantation site. In many instances it would be preferable to use either a flat disc or a thin cylinder. In the case of testosterone, the need of large active “releasing” surface area to be able to provide with the required dose makes disc-like capsules particularly suitable.

In one embodiment, the device or capsule does not comprise a filter for separating the pharmaceutical substance of solid state from the nanochannels. In one embodiment, the capsule does not comprise any filter.

In one embodiment, after implantation, the device or capsule can be reloaded without having to be explanted first. In other words, the capsule can be reloaded while remaining in the body of the patient. In one embodiment, the composition to be reloaded comprises the same pharmaceutical substance as the original. In another embodiment, the composition to be reloaded comprises a pharmaceutical substance different from the original.

For example, solid or polymeric testosterone or Levothyroxine formulation can be reloaded into the nanochannel implant without need of explanting the device form the body. This can be achieved through the use of two injection ports, which are recessed with respect of the capsule surface for an easier determination of their position through the skin One port can be used for loading while the other for flushing and venting of previously contained material within the implant. Both powder and polymeric formulation can be prepared in a “fluid paste” state that can be inserted by applied pressure into the capsule cavity while vacuuming from the venting needle. In the case of powder the paste can contain the smallest amount of liquid necessary to reduce the viscosity/friction within the injection needle. In the case of solid polymeric formulation, the polymer can be injected into the capsule in its pre- polymerized status and allowed to polymerize within the implant. The polymerization can be associated to an exothermic reaction which produces heat. A tolerable level of heat, compatible with such application, can be easily obtained by tuning the polymeric formulation.

### Solid State Pharmaceutical Substance and Solution/Solvent

Heterogenous mixtures and compositions can comprise more than one phase. For example, a solid phase can be in contact with a solvent or a solution. The solution in contact with the solid can be a saturated or supersaturated solution, continuously dissolving the solid. For purposes herein, a supersaturated solution is an example of a type of saturated solution.

The capsule described above can be loaded with a composition comprising at least one pharmaceutical or therapeutical substance of solid state. The composition can comprise, for example, a solvent. The presence of the solvent helps to create a continuity of fluids throughout the membrane, connecting the body with the inside of the capsule. The solvent can comprise, for example, less than 80 wt. % of the composition, or less than 70 wt. % of the composition, or less than 60 wt. % of the composition, or less than 50 wt. % of the composition, or less than 40 wt. % of the composition, or less than 30 wt. % of the composition, or less than 20 wt. % of the composition, or less than 10 wt. % of the composition, or less than 5 wt. % of the composition, or less than 2 wt. % of the composition, or less than 1 wt. % of the composition. The amount of solvent can be, for example, sufficient to wet the pellet to a desired amount, which can be one surface of the pellet or the entire rod.

The optional solvent can dissolve, for example, less than 80 wt. % of the pharmaceutical or therapeutical substance, or less than 70 wt. % of the pharmaceutical or therapeutical substance, or less than 60 wt. % of the pharmaceutical or therapeutical substance, or less than 50 wt. % of the pharmaceutical or therapeutical substance, or less than 40 wt. % of the pharmaceutical or therapeutical substance, or less than 30 wt. % of the pharmaceutical or therapeutical substance, or less than 20 wt. % of the pharmaceutical or therapeutical substance, or less than 10 wt. % of the pharmaceutical or therapeutical substance, or less than 5 wt. % of the pharmaceutical or therapeutical substance, or less than 2 wt. % of the pharmaceutical or therapeutical substance, or less than 1 wt. % of the pharmaceutical or therapeutical substance.

Any pharmaceutical or therapeutical substance of solid state at ambient condition can be used with the methods described here. In one embodiment, the pharmaceutical or therapeutical substance is a chemotherapy drug. In another embodiment, the pharmaceutical or therapeutical substance is a sex hormone such as testosterone. In a further embodiment, the pharmaceutical or therapeutical substance is a thyroid or thyroid-related hormone such as thyroxine. Other examples include diabetic drugs and cholesterol lowering drugs.

In one embodiment, the pharmaceutical or therapeutical substance may have a low aqueous solubility, making it unsuitable for delivering as a liquid formulation in a small implantable capsule. For example, the aqueous solubility of the pharmaceutical or therapeutical can be 500 mg/ml or less, or 100 mg/ml or less, or 10 mg/ml or less, or 1 mg/ml or less, or 100 μg/ml or less, or 10 μg/ml or less. In another embodiment, the pharmaceutical or therapeutical substance has a relatively high aqueous solubility but has to be delivered in large quantity into a patient to be effective, making it also unsuitable for delivering as a liquid formulation in a small implantable capsule. In a further embodiment, the pharmaceutical or therapeutical substance is loaded in solid form to improve loading efficiency, drug stability and/or duration of treatment.

The pharmaceutical or therapeutical substance can be in any solid form, such as pellet, powder, crystal, nanoparticles, microparticles, degradable polymer, liposome, emulsion, etc.

The composition described here can further comprise one or more pharmaceutically acceptable carrier including excipients, diluents, adjuvants, stabilizers, emulsifiers, preservatives, colorants, buffers, flavor imparting agents, absorption enhancers, complexing agents, solubilizing agents, wetting agents and/or surfactants.

The composition can comprise, for example, only one pharmaceutical or therapeutical substance of solid state partially dissolved in a solvent. The composition can comprise, for example, only one pharmaceutical or therapeutical substance of solid state in absence of any solvent. The composition can comprise, for example, two or more pharmaceutical or therapeutical substances both of solid state and both partially dissolved in a solvent. The composition can comprise, for example, two or more pharmaceutical or therapeutical substances of solid state and in absence of any solvent. The composition can comprise, for example, a first pharmaceutical or therapeutical substance totally dissolved in a solvent and a second pharmaceutical or therapeutical substance of solid state partially dissolved in a solvent.

### Implantation of Capsule

Methods for implanting drug-delivery devices into patients are known in the art and described in, for example, US 2006/0252049; US 2006/0008512; US 2011/0046606; and Prescott et al., Nature Biotechnology 24(4):437- 438 (2006), all of which are incorporated herein by reference in their entireties.

### Release of Pharmaceutical Substance

Methods for zero-order delivery of liquid state pharmaceutical substances are known in the art and described in, for example, Grattoni et al., Lab on a Chip 10:3074-3083 (2010); Grattoni et al., Pharm. Res. 28(2):292-300 (2011); Grattoni et al., Anal. Chem. 83:3096-3103 (2011); Walczak et al., Nanobiotechnology 1:35-42 (2005); Cosentino et al., J. Phys. Chem. 109:7358-7364 (2005); and Ziemys et al., Journal of Computational Physics 230:5722-5731 (2011), all of which are incorporated by reference in their entireties. However, methods for delivering solid state pharmaceutical substances are lacking.

Methods described here are capable of a constant and sustained delivery of pharmaceutical substance of solid state for an extended period of time. In a preferred embodiment, the implanted capsule described here are capable of achieving substantially zero-order delivery of the pharmaceutical substance. For example, the pharmaceutical substance can be released at about the same rate for at least 3 months, or at least 6 months, or at least 9 months, or at least 12 months, or at least 18 months, or at least 24 months, or at least 30 months, or at least 36 months.

The release rate of the pharmaceutical substance can be, for example, within the scope of the effective dose thereof in a patient. The pharmaceutical substance can be released at a rate of, for example, about 1-10 μg/day, or about 10-100 μg/day, or about 100-1,000 μg/day, or about 1-10 mg/day, or about 10-100 mg/day.

Notwithstanding the different effective release rates of different pharmaceutical substances, capsules described here are capable of loading a sufficient quantity of a given solid state substance for substantially zero-order release of at least 6 months, or at least 9 months, or at least 12 months, or at least 18 months, or at least 24 months, or at least 30 months, or at least 36 months. The rate of said zero-order release is within the effective ranges of said pharmaceutical substance in human beings of different ages.

### Additional Embodiments

As shown in FIG. 1C, one embodiment of the methods described here utilizes an implantable nanochannel device for the sustained and constant administration of molecules and therapeutics (e.g. hormones or drugs), which are contained in the implant reservoir in a variety of formulations, such as solid, semi-solid, liquid, emulsion, liposome, polymer, nanoparticles, microparticles, powder and crystal. In a preferred embodiment, a solid state pharmaceutical substance is delivered. The implant reservoir, the shape of which is optimized for the type of drug, needed release rate and anatomically desired location of the implant, contains the therapeutic agent in a solid state and a small volume of solvent. The size and shape of the implant can be altered to accommodate a broad range. The solid drug is dissolved over time in the solvent establishing a concentration, which may reach the solubility limit. The drug then diffuses through the nanochannel membrane, which allows maintaining a concentration independent release due to the nanoconstraint properties exerted on the drug molecules. Such method allows for the constant release of drugs and therapeutics for periods ranging from weeks to years. By storing drug in a solid state the implant maximizes the loading efficiency, minimizing the reservoir volume per unit mass of drug, and maximizes the drug stability over time. The nanochannel membrane operates as a system that neutralizes initial drug release ‘burst’ and decreasing release profiles, common limiting factors of solid degrading drug formulations (e.g. implantable pellets, degradable polymers).

Methods and devices described here would improve the loading efficacy of the implant and the stability of drugs and therapeutics over time, making the payload suitable for long-period treatments (from months to years). This innovative system broadens the use of the nanochannel delivery membrane for the sustained and constant release of molecules presenting very low solubility including a large number of chemotherapeutics and hormones among other drugs. Moreover, it allows for the development of improved hormone delivery/replacement technologies to deliver a basal and constant amount of hormones for the treatment of chronic pathologies. Furthermore, by employing a solid drug formulation, this invention solves the possible issue of overdosing the patient in the remote case of implant rupture. Still further, a reloadable device can be used to refill the reservoir without explantation.

Table 1-3 below depict calculated amounts of thyroxine, volumes of thyroxine together with solvent, and sizes of implant device corresponding to different desired release rates and durations of treatment. For example, a patient who needs 100 μg/day for 1 year would require a device holding 36 mg in 38.4 μl with the device measuring 11.6 mm in diameter. These configurations are readily achievable and practical for clinical use. In practice, patients can take oral thyroxine first to determine the optimal daily dose and then convert to a longer acting implant as described.

TABLE 1
Total amount of levothyroxine (mg) required for the treatment

| Delivery | 6 (duration | 12 (duration | 24 (duration | 36 (duration |
|---|---|---|---|---|
| Rate | of therapy- | of therapy- | of therapy- | of therapy- |
| (μg/day) | months) | months) | months) | months) |
| 50 | 9 | 18 | 36 | 54 |
| 100 | 18 | 36 | 72 | 108 |
| 150 | 27 | 54 | 108 | 162 |
| 200 | 36 | 72 | 144 | 216 |
| 250 | 45 | 90 | 180 | 270 |
| 300 | 54 | 108 | 216 | 324 |
| 400 | 72 | 144 | 288 | 432 |

TABLE 2
Volume of levothyroxine (μl) for the entire treatment

| Delivery | 6 (duration | 12 (duration | 24 (duration | 36 (duration |
|---|---|---|---|---|
| Rate | of therapy- | of therapy- | of therapy- | of therapy- |
| (μg/day) | months) | months) | months) | months) |
| 50 | 9.6 | 19.2 | 38.4 | 57.6 |
| 100 | 19.2 | 38.4 | 76.8 | 115.2 |
| 150 | 28.8 | 57.6 | 115.2 | 172.8 |
| 200 | 38.4 | 76.8 | 153.6 | 230.4 |
| 250 | 48 | 96 | 192 | 288 |
| 300 | 57.6 | 115.2 | 230.4 | 345.6 |
| 400 | 76.8 | 153.6 | 307.2 | 460.8 |

TABLE 3
Estimated implant diameter (mm) for disc-like implant
(diameter to thickness ratio = 5)

| Delivery | 6 (duration | 12 (duration | 24 (duration | 36 (duration |
|---|---|---|---|---|
| Rate | of therapy- | of therapy- | of therapy- | of therapy- |
| (μg/day) | months) | months) | months) | months) |
| 50 | 10.3 | | | |
| 100 | | 11.6 | | |
| 150 | | | 14.5 | |
| 200 | | | | 17.7 |
| 250 | | | | |
| 300 | | | | |
| 400 | | | | 23 |

### WORKING EXAMPLES

Additional embodiments are provided in the following non-limiting working examples.

### Example 1

### Device and Measurement

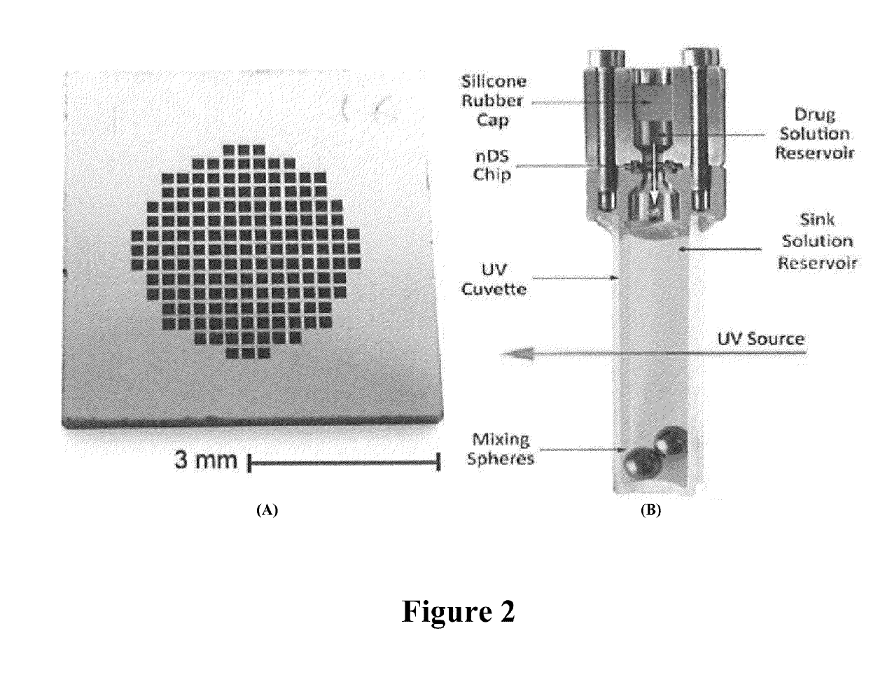

The implantable capsule comprising one or more nanochannel membranes were fabricated according to US 2010/0152699 and PCT/US2009/064376. The nanochannel membranes present nanochannels ranging in sizes between 3 and 50 nm. The nanochannel membranes (FIG. 2A) were produced in 29 configurations presenting different constant rates of delivery. Custom diffusion devices (FIG. 2B) were utilized for measuring the amount of drug released from nanochannel membrane devices using UV-spectroscopy, as described in Grattoni et al., Lab on a Chip 10:3074-3083 (2010) and Grattoni et al., Anal. Chem. 83:3096-3103 (2011). A linear standard curve relating UV absorbance and concentration of diffused substance was obtained at a wavelength of 250 nm and used for the release test of testosterone. A linear standard curve relating UV absorbance and concentration of diffused substance was obtained at a wavelength of 240 nm and used for the release test of levothyroxine.

### Example 2

### Constant Delivery of Testosterone

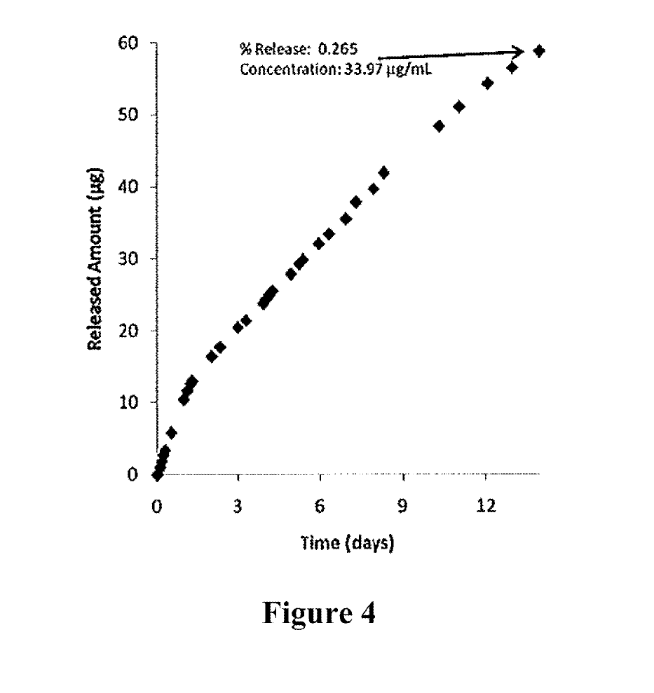

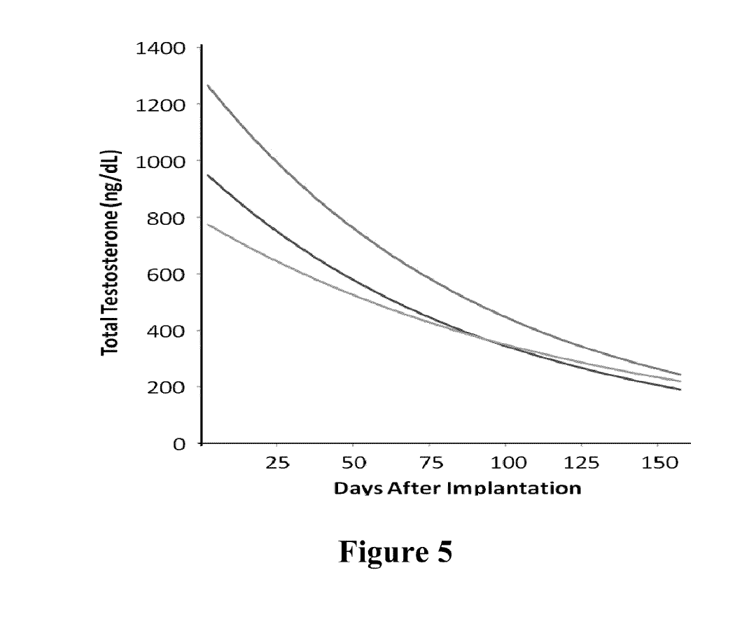

In a preliminary experiment, the nanochannel implant was loaded with degradable testosterone pellets immersed in DI water. The amount of DI water inserted in the capsule was approximately 700 μL. Constant release of testosterone was achieved from solid pellets (containing 24 mg of testosterone) through 3 nm nanochannel membrane at the release rate of 5 μg/day for 13 days (FIG. 4). In comparison, the release profile of degradable testosterone pellets alone includes not only a burst release, but also a constant decay of the release rate (FIG. 5).

In subsequent long-term tests, testosterone pellet and powder formulations were loaded into implantable capsules comprising 3 nm nanochannel membrane and implantable capsules comprising 40 nm nanochannel membrane, respectively, to test the long-term release of testosterone.

In the first long-term testosterone release experiment, two testosterone formulations, powder and pellet (Testopel), were used. The release experiment was performed from a capsule into a bottle containing the recipient sink solution. A 3 nm membrane was used for each testosterone formulation and assembled within the capsule. Each nanochannel presented a width of 5 μm and a length of 3 μm. The nanochannel membrane was fabricated by NanoMedical Systems, Inc., Austin Tex. by using the fabrication methods as described in PCT/US2009/064376. In general, the nanochannel membrane was fabricated with microfabrication techniques by employing a sacrificial layer technique, to obtain precise nanochannels parallel to the membrane surface and connected to the membrane inlet and outlet by means of sets of microchannels.

The total number of nanochannel per membrane is equal to 118496. Testosterone powder (21.2 mg) was weighted into the titanium capsule, which was filled with 791 μL of Millipore water. The second titanium capsule was loaded with of 21.9 mg of the pellet and 882.8 μl of Millipore water. Each capsule was dropped into a glass bottle, which was filled with 25 mL of Millipore water and stirred with a magnetic bar for homogeneity of the solution. Both bottles were kept in a dark, 37° C. incubator. For the release measurement of both testosterone conformations, a UV-Vis absorbance versus concentration standard curve was prepared. The testosterone solutions were sampled every other day and the UV absorbance was measured at a wavelength of 250 nm. The sampling method consisted of removing 1.5 mL of the sink solution, measuring the absorbance, and returning the sample to the bottle. To prevent the saturation of the sink solution, the whole 25 mL of Millipore water was replaced at regular intervals with fresh solvent.

The second long-term testosterone release experiment was run with the same setup and measurement method. In this case, 40 nm membranes with 8 μm wide and 1 μm long nanochannels were used for both testosterone formulations. The nanochannel membrane was fabricated by NanoMedical Systems, Inc., Austin Tex. by using the fabrication methods as described in PCT/US2009/064376. In general, the nanochannel membrane was fabricated with microfabrication techniques by employing a sacrificial layer technique, to obtain precise nanochannels parallel to the membrane surface and connected to the membrane inlet and outlet by means of sets of microchannels.

Testosterone powder and pellet (26 and 24.7 mg, respectively) were weighted into PEEK capsules. Both capsules were filled with 900 μL and dropped into 90 mL of Millipore water. The sampling measurement was performed every other day with the same method described above.

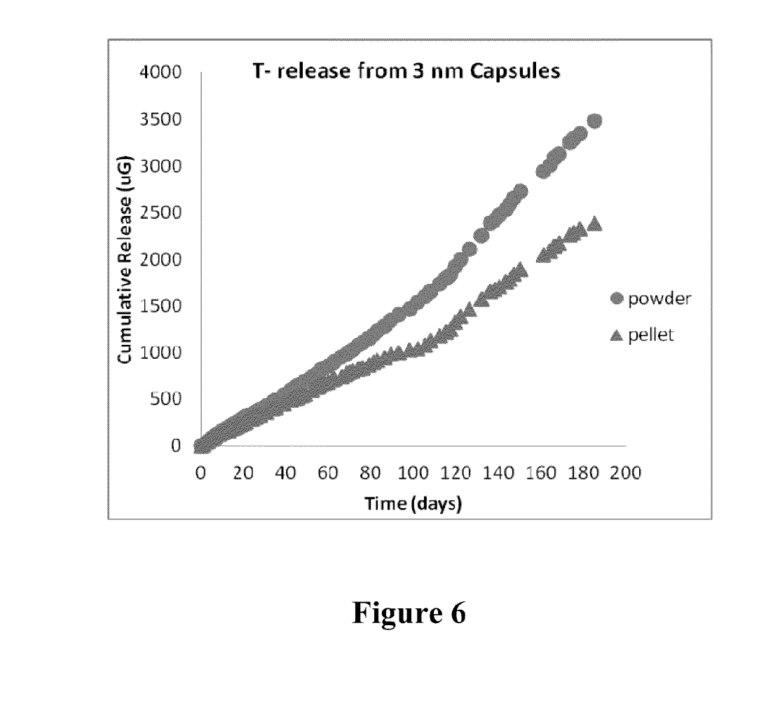

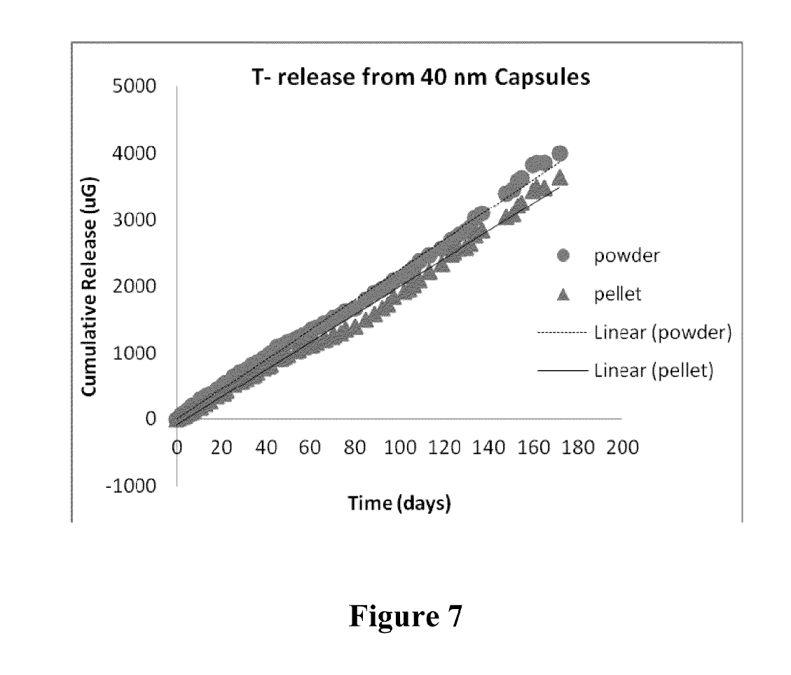

As shown in FIG. 6, implantable capsules comprising 3 nm nanochannel membrane are capable of achieving linear delivery of testosterone for at least 180 days, whether the testosterone is in a pellet formulation (Testopel) or a powder formulation. As shown in FIG. 7, implantable capsules comprising 40 nm nanochannel membrane are capable of achieving linear delivery of testosterone for at least 160 days, whether the testosterone is in a pellet formulation (Testopel) or a powder formulation.

### Example 3

### Constant Delivery of Levothyroxine

In this experiment, L-thyroxine sodium pentahydrate (Sigma Aldrich, Cat. No. T2501) was released from a bottle-capsule setup as described in the case of testosterone. Two 3 nm membranes with 3 μm wide and 1 μm long nanochannels were used. The nanochannel membrane was fabricated by NanoMedical Systems, Inc., Austin Tex. by using the fabrication methods as described in PCT/US2009/064376. In general, the nanochannel membrane was fabricated with microfabrication techniques by employing a sacrificial layer technique, to obtain precise nanochannels parallel to the membrane surface and connected to the membrane inlet and outlet by means of sets of microchannels

PEEK capsules were loaded with 852 and 875 μL of Millipore water and 17.1 mg of powder. Each capsule was dropped into a glass bottle filled 50 mL of Millipore water and stirred with a magnetic bar for homogeneity of the solution. Both bottles were kept in a dark, 37° C. incubator. For the release measurement, a UV-Vis absorbance versus concentration standard curve was prepared. The absorbance was measured at 240 nm. The sampling method consisted of removing 1.5 mL of the sink solution, measuring the absorbance, and returning the sample to the bottle. To prevent the saturation of the sink solution, the whole 50 mL of Millipore water was replaced at regular intervals with fresh solvent.

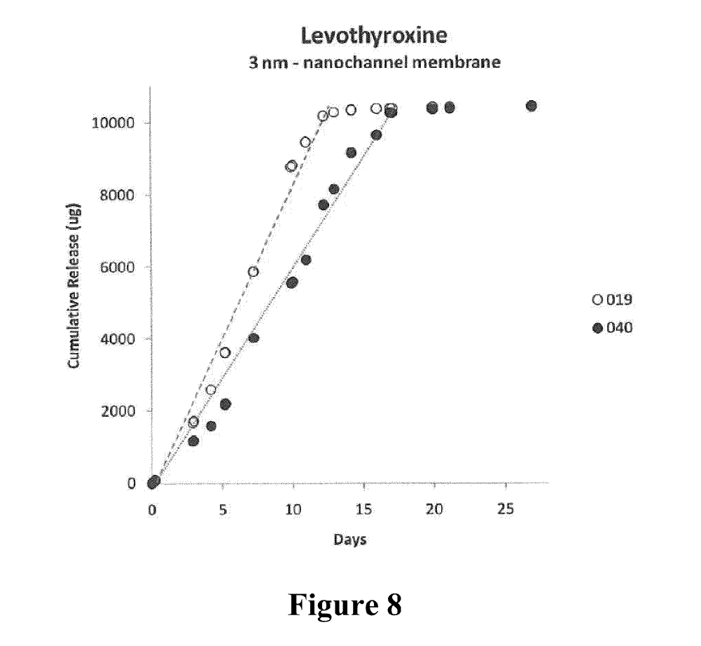

As shown in FIG. 8, continuous and constant release of levothyroxine was achieved over 17 days in 2 separate experiments (i.e., 019, 040). This release pattern corresponds to an approximate daily release rate of 500 μg/day. Current conventional human dosing is 50-400 μg/day.

1. An implantable device comprising:
at least one implant body;
at least one reservoir in said implant body;
wherein inside the reservoir is disposed at least one pharmaceutical substance in a solid state contacted by at least one solution of said pharmaceutical substance, said solution comprising at least one solvent, and wherein said pharmaceutical substance in the solid state is in a form other than that of a suspension; and
at least one nanochannel membrane for delivering said pharmaceutical substance from the reservoir to a patient.

2. The implantable device of claim 1, wherein the implant body comprises at least one exit port; wherein the pharmaceutical substance is testosterone in powder or pellet form; wherein the nanochannel membrane comprises at least one nanochannel having at least one lateral dimension of 1-200 nm; and wherein the nanochannel membrane is in fluid communication with the reservoir and the exit port to provide delivery of the testosterone from the reservoir to the exit port.

3. The implantable device of claim 1, wherein the implant body comprises at least one exit port; wherein the pharmaceutical substance is thyroxine in powder or pellet form; wherein the nanochannel membrane comprises at least one nanochannel having at least one lateral dimension of 1-200 nm; and wherein the nanochannel membrane is in fluid communication with the reservoir and the exit port to provide delivery of the testosterone from the reservoir to the exit port.

4-5. (canceled)

6. The implantable device of claim 1, wherein the implant body comprises at least one exit port.

7. The implantable device of claim 1, wherein the implant body comprises at least one removable cap comprising at least one exit port for delivery of the pharmaceutical substance to the patient and in fluid communication with the nanochannel membrane.

8. The implantable device of claim 1, wherein said pharmaceutical substance in the solid state is a chemotherapy drug or a hormone.

9. (canceled)

10. The implantable device of claim 1, wherein said pharmaceutical substance in the solid state is in the form of pellet or powder.

11. (canceled)

12. The implantable device of claim 1, wherein said solvent represents less than 50 wt. % of the composition.

13. The implantable device of claim 1, wherein said solvent dissolves less than 50 wt. % of the total amount of said pharmaceutical substance in said composition.

14. The implantable device of claim 1, wherein said solution is a saturated solution.

15-16. (canceled)

17. The implantable device of claim 1, wherein said silicon nanochannel membrane comprises at least 100,000 nanochannels each having at least one lateral dimension of 3-50 nm.

18. The implantable device of claim 1, wherein said nanochannel membrane further comprises at least one inlet microchannel and at least one outlet microchannel, wherein the inlet microchannel is in fluid communication with the outlet microchannel via the nanochannel, wherein the nanochannel membrane is oriented parallel to the primary plane of the nanochannel membrane, and wherein a flow path from the inlet microchannel to the nanochannel to the outlet microchannel requires a maximum of two changes in direction.

19. The implantable device of claim 1, wherein said capsule does not comprise a filter for separating said pharmaceutical substance of solid state from said nanochannels.

20. The implantable device of claim 1, wherein the implant body comprises at least one removable cap comprising at least one exit port for delivery of the pharmaceutical substance to the patient and in fluid communication with the nanochannel membrane.

21. The implantable device of claim 1, wherein the device provides for drug delivery for at least 365 days.

22. (canceled)

23. A method for delivering a pharmaceutical substance, comprising:
providing the implantable device according to claim 1, and implanting said device into a patient, wherein said pharmaceutical substance is released from the device to contact said patient, and wherein pharmaceutical substance is released in zero-order fashion for at least 6 months.

24. A method for delivering testosterone, comprising:
providing the implantable device according to claim 2, and
implanting said device into a patient, wherein the testosterone is released from the device to the patient at a rate of 1-10 mg/day.

25. A method for delivering thyroxine, comprising:
providing the implantable device according to claim 3, and
implanting said device into a patient, wherein the thyroxine is released from the device to the patient at a rate of 50-400 μg/day.

26. (canceled)

27. The method of claim 23, wherein implanting said device into said patient is not followed by a burst release of said pharmaceutical substance, and wherein said pharmaceutical substance is released in zero-order fashion for at least 12 months.

28. The method of claim 23, further comprising reloading said device with said pharmaceutical substance after implantation, wherein said reloading step does not comprise explanting said capsule from said patient.

29-30. (canceled)
